(19) United States
(12) Patent Application Publication
(10) Pub. No.: US 2025/0001155 A1
(43) Pub. Date: Jan. 2, 2025

(54) FLUID CONNECTOR ASSEMBLY

(71) Applicant: CareFusion 303, Inc., San Diego, CA (US)

(72) Inventors: Abin AUSTIN, Thrissur (IN); Jason Andrew WINE, Brea, CA (US); Amarsinh Deeliprao JADHAV, Hyderabad (IN); Mohammed Mehtab KHAN, Bengaluru (IN); Kanjimpuredathil Muralikrishna MENON, Bangalore (IN)

(21) Appl. No.: 18/342,089

(22) Filed: Jun. 27, 2023

(51) Int. Cl. A61M 39/10; A61M 39/14

(52) U.S. Cl. CPC A61M 39/1011; A61M 39/14; A61M 2039/1027

(57) ABSTRACT

Fluid connector assemblies that seal off fluid paths in the respective connectors are disclosed. When connectors of a fluid connector assembly are connected to each other, respective compressible members in the connectors are displaced, allowing downstream fluid passage through the fluid connector assembly. The connectors may be coupled via a connecting mechanism that that provides a threshold retention force. When an external force greater than the threshold force is applied to the fluid connector assembly, the snap mechanism may no longer maintain the connectors together, causing the connectors to decouple from each other.

### TECHNICAL FIELD

The present disclosure relates generally to medical fluid connectors and, more particularly, to a fluid connector assembly that includes medical connectors that decouple with each other due to an applied force, with each medical connector designed to automatically seal off their respective fluid paths. The decoupling may be due to intentional or unintentional separation between the medical connectors.

### BACKGROUND

Peripheral intravenous (“PIVC”) catheters are medical tools inserted into peripheral veins of patients to deliver medical fluid to the patients. In an example application, the medical fluid is delivered to the patient, and a medical professional subsequently removes the PIVC catheter from the patient. Often, however, these catheters are unintentionally dislodged. For example, catheter lines receiving an unintended or unexpected pulling force can pull the IV tubing, which pulls the catheter out of the patient. In other instances, catheters are accidentally removed from patients and medical professionals. Unintended or unexpected dislodgement can lead to patient blood loss, IV fluid loss, and IV fluid delivery delay.

### SUMMARY

In accordance with at least some embodiments disclosed herein is the realization that unintended dislodgement or disconnection of a medical connection, such as a medical fluid line, can result in injury to a patient or a medical professional, such as by depriving the patient of a medicament, increasing the potential for infection to the patient, and exposing the medical professional to medicaments.

Aspects of the present disclosure provide fluid connector assemblies with medical connectors, each of which include one or more fluid paths, that respond to unintentional or unexpected external forces by decoupling from each other and sealing off their respective fluid paths. The decoupling may include automatic decoupling using bellows or other elastically compressible member that decompress and return to their original shape external forces are no longer acting upon them. Beneficially, fluid connector assemblies described herein can limit or prevent patient blood loss, IV fluid loss, infection, and medical delivery delays. Further, aspects of the present disclosure provide connecting mechanisms that aid in the connection of the fluid connector assembly in the event that the fluid connector assembly is decoupled.

According to certain embodiments, a fluid connector assembly includes a first connector, a second connector, and a connecting mechanism. The second connector may be configured to couple with the first connector and may include a snap member disposed on an end thereof. The connecting mechanism may be coupled to the second connector for detachably coupling the first connector and the second connector. The connecting mechanism may include a body having a channel extending therethrough for providing fluid communication between the first connector and the second connector, and at least one projection extending from an end of the body. When the connecting mechanism is coupled to the second connector, at least a portion of the connecting mechanism may be positioned within an opening of the snap member and secured by the projection. Moreover, actuation of the snap member may allow the connecting mechanism to be disconnected from the second connector.

In some embodiments, the connecting mechanism remains coupled to the second connector by a threshold force and, wherein, an external force greater than the threshold force is applied to at least one of the first connector and the second connector, the connecting mechanism is decoupled from the second connector. The external force may be a pulling force of at least five pounds.

In some embodiments, the snap member includes at least one snap arm extending from an end of the snap member and wherein actuation of the at least one snap arm allows for the connecting mechanism to be decoupled from the second connector.

In some embodiments, the connecting mechanism further includes a valve member disposed within the channel. When the first connector and the second connector are coupled, the valve member may be configured to allow fluid communication between the first connector and the second connector. When the first and second connectors are decoupled, the valve member may be configured to prevent a flow of a fluid through the channel.

In some embodiments, the projection extends outwardly with respect to a longitudinally extending central axis and an exterior surface profile of the projection corresponds to an interior profile of the opening of the snap member.

In some embodiments, at least a portion of the connecting mechanism couples to an exterior surface of the second connector. When a pulling surface greater than a threshold force is applied to the second connector in a direction along a central longitudinal axis, the connecting mechanism may actuate the snap member thereby resulting in the first connector and second connector being decoupled and at least a portion of the connecting mechanism remains coupled to the second connector when the first connector and the second connector are decoupled.

In some embodiments, the fluid connector assembly is a closed-system drug transfer device.

According to certain embodiments, a method of coupling a fluid connector connecting assembly includes providing a first connector, a second connector configured to couple to the first connector, and a connecting mechanism configured to couple the first and second connectors. The second connector may include a snap member disposed on an end thereof. The connecting mechanism may include a body and at least one projection extending from an end of the body. The method may include coupling an end of the connecting mechanism to the second connector such that at least a portion of the connecting mechanism is disposed within an opening of the snap member; and coupling the first connector to an opposite end of the connecting mechanism such that the first connector, the second connector, and the coupling mechanism are in fluid communication.

In some embodiments, coupling the end of the connecting mechanism to the second connector includes positioning the projection within the opening of the snap member such that an exterior surface of the at least one projection abuts an interior surface of the snap member in a snap fit configuration. Moreover, decoupling the connecting mechanism from the second connector may include actuating the snap member to provide clearance between the at least one projection and the interior surface thereby allowing the connecting mechanism to be removed from the opening.

In some embodiments, the connecting mechanism remains coupled to the second connector by a threshold force and, when an external force greater than the threshold force is applied to at least one of the first connector and the second connector, the connecting mechanism is decoupled from the second connector.

In some embodiments, the connecting mechanism further comprises a valve member disposed within a channel formed within the body of the connecting mechanism. When the first connector and the second connector are coupled, the valve member may be configured to allow fluid communication between the first connector and the second connector. When the first and second connectors are decoupled, the valve member may be configured to prevent a flow of a fluid through the channel.

According to certain embodiments, a fluid connector assembly includes a first connector, a second connector configured to couple to the first connector, and a connecting mechanism for detachably coupling the first connector and the second connector. The second connector may be a closed system having a snap member disposed having at least one arm projecting from an end of the snap member. The connecting mechanism may include a body having a channel extending therethrough for providing fluid communication between the first connector and the second connector, a valve member disposed within the channel for controlling a flow of fluids through the channel, a first projection extending from a first end of the body for coupling to the first connector, and a second projection extending from a second end of the body for coupling to the second connector. When the connecting mechanism is coupled to the second connector, at least a portion of the connecting mechanism may be positioned within an opening of the snap member and secured by the second projection. When a separating force exceeds a predetermined threshold, the connecting mechanism may be decoupled from the second connector.

In some embodiments, the predetermined threshold is a pulling force of at least five pounds.

In some embodiments, the arm includes a first snap arm projecting from a first side of the second connector and a second snap arm projecting from a second side of the second connector opposite the first side. Furthermore, actuation of the first snap arm and the second snap arm may allow for the connecting mechanism to be decoupled from the second connector.

In some embodiments, when the first connector and the second connector are coupled, the valve member is configured to allow fluid communication between the first connector and the second connector, and, when the first connector and the second connector are decoupled, the valve member is configured to prevent a flow of a fluid through the channel.

In some embodiments, each of the first projection and the second projection extends outwardly with respect to a longitudinally extending central axis of the connecting mechanism. Furthermore, an exterior surface profile of the second projection may correspond to an interior profile of the opening of the snap member.

In some embodiments, the connecting mechanism includes a ring member slidably disposed on an exterior surface of the second connector and configured to actuate the snap member.

Furthermore, when a separating force exceeds a predetermined threshold, the ring member may slide along the second connector until the ring member is disposed on the at least one arm thereby causes actuation of the snap member.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an IV set coupled to a patient, in accordance with aspects of the present disclosure.

FIG. 2 illustrates a partial cross-sectional view of the connector of the fluid connector assembly, in accordance with aspects of the present disclosure.

FIGS. 3A-3B illustrate a partial cross-sectional view of the connector of the fluid connector assembly, in accordance with aspects of the present disclosure.

FIG. 4 illustrates a partial cross-sectional view of the fluid connector assembly, in accordance with aspects of the present disclosure.

FIGS. 5A-5C illustrate partial cross-sectional views of a connecting mechanism of the fluid connector assembly, in accordance with aspects of the present disclosure.

FIG. 6 illustrates a partial cross-sectional view of the fluid connector assembly, in accordance with aspects of the present disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present disclosure includes various features and advantages of a fluid connector assembly with medical connectors, for example a closed-system drug transfer devices (CSTD), that seal off their respective fluid paths when the medical connectors are decoupled from each other. Additionally, the fluid connector assembly may be formed so as to prevent the transfer of environmental contaminants into the system as well as prevent the escape of a hazardous drug or vapor concentrations from the device. The medical connectors may each include a compressible member that decompresses in response to the decoupling, thus providing an automatic sealing of the respective fluid paths. The fluid connector assembly may further include connecting mechanisms, for connecting components of the fluid connector assembly while also maintaining the closed system of the assembly. Moreover, the connecting mechanisms may be configured to disconnect from the connectors of the fluid connector assembly when a threshold pull force is reached while preventing environmental contaminants into the system and also preventing the escape of the contained drug or vapor thereby preserving the closed-system.

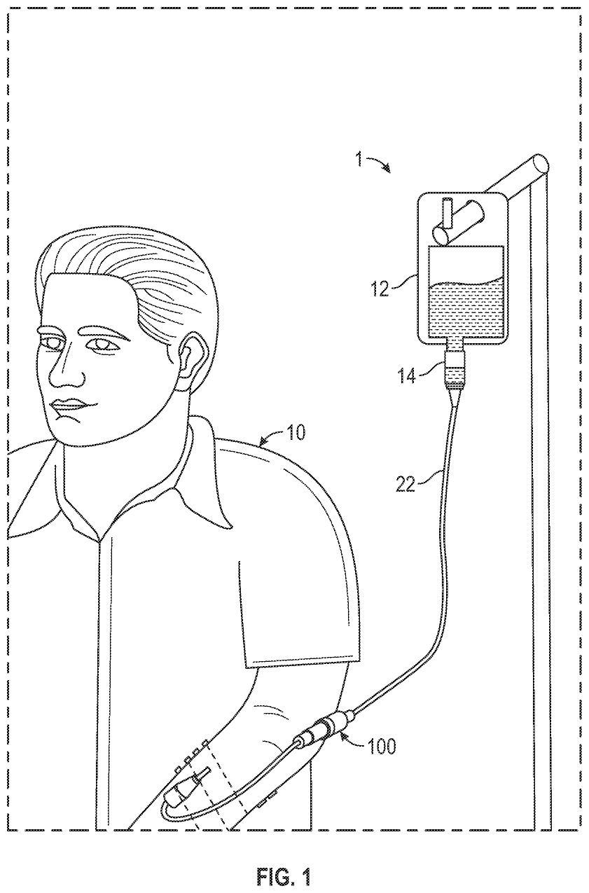

Referring now to the figures, FIG. 1 illustrates an IV set 1 coupled to a patient 10, in accordance with aspects of the present disclosure. The IV set 1 includes a medicament bag 12, a drip chamber 14, and tubing 22. The tubing 22 extends between the drip chamber 14 and a fluid connector assembly 100 of the IV set 1. To resist unintended dislodgement or disconnection of the tubing 16 or the catheter 18 from the patient, tape 26 is placed over the tubing 16 and the catheter 18, so that the tape 26 engages the tubing 16, the catheter 18, and the patient 10.

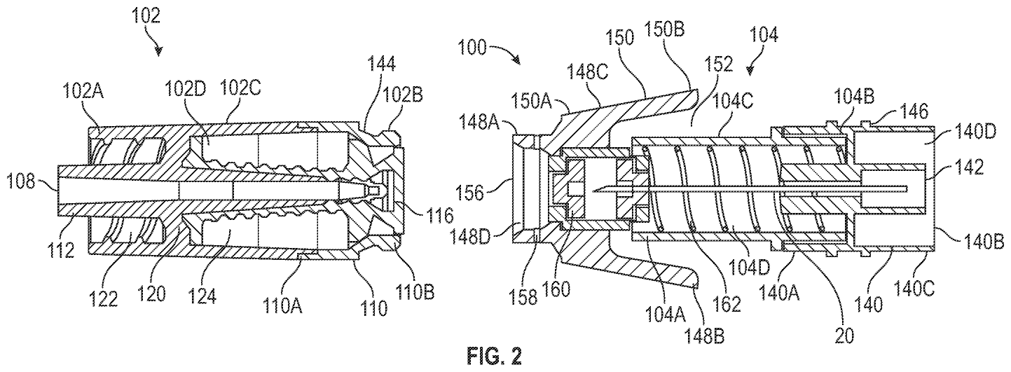

FIG. 2 illustrates a perspective view of a fluid connector assembly 100, in accordance with aspects of the present disclosure. The fluid connector assembly 100 is designed for use in medical applications, such as the IV set 1 (shown in FIG. 1) as well as other IV medical fluid delivery applications using catheters, including PIVC catheters, as non-limiting examples.

As shown, the fluid connector assembly 100 includes a connector 102 and a connector 104 coupled with the connector 102. The connector 102 and the connector 104 may be referred to as a first connector and a second connector, respectively. However, “first” and “second” may be interchangeable. Also, each of the connectors 102 and 104 may be referred to as medical connectors. When the connectors 102 and 104 are connected to each other as shown in FIG. 2, a fluid path for medical fluid is established by the fluid connector assembly 100.

In some embodiments, the connector 104 is connected to a medical fluid (not shown). Further, in some embodiments, the connector 102 is connected to a catheter line (not shown) that delivers the medical fluid to a catheter. In this regard, the connector 104 may include a fluid inlet 106 that acts as a fluid receiving location for the fluid connector assembly 100. Also, the connector 102 may include a fluid outlet 108 that acts as a fluid transmission location for the fluid connector assembly 100.

To facilitate the connection to the medical fluid, the connector 104 may include a luer. In some embodiments, the luer is female luer designed to mate with a male connector that is connected to the medical fluid. Similarly, to facilitate the connection to the catheter line, the connector 102 may include a luer. In some embodiments, the luer is male luer designed to mate with a female connector that is connected to the catheter line. Each of the luers may conform to standards established by the International Organization for Standards (“ISO”) to improve patient safety, minimize medical fluid leakage, and reduce misconnection with other connection devices.

Additionally, the connector 102 may include a post 112 passing centrally, or at least approximately centrally, through the connector 102. The post 112 may include a channel 114 that establishes the fluid outlet 108. While the post 112 is illustrated as cylindrical, the post 112 need not be so limited and may have any suitable.

The connector 102 may include a housing integrated with the luer. The connector 102 may include a first end 102A having the integrated luer and a second end 102B opposite the first end 102A and configured to couple to a receiving ring 110. The second end 102B have a stepped profile with a protruding portion having a reduced thickness relative the thickness of the rest of the housing.

The connector 102 may include an exterior surface 102C and an interior surface 102D. The connector 102 may include a hollow, or generally hollow, body that carries one or more components. For example, in addition to the post 112, a compressible member 116 may be housed within the connector 102. The connector 102 may further include a wall 120 extending laterally across the interior of the connector 102 thereby separating the interior of the connector 102 into separate cavities 122, 124. The post 112 may be integrally formed with the wall 120 and extend through each of the cavities 122, 124. The post 112 may include an open first end 120A, a closed second end 120B opposite the first end 120A, and the channel 114 extending through the post 112. The first end 120A may include an opening for the channel 114, which forms the fluid outlet 108. The second end 120B of the post 112 may be a substantially closed end and include one or more apertures, also referred to as flow windows, 128.

The connector 102 may further include the receiving ring 110 disposed at, and coupled to, the second end 102B of the connector 102. The receiving ring 110 may include a first end 110A configured to be coupled to the second end 102B and a second end 110B opposite the first end 110A. The first end 110A of the receiving ring 110 may have a stepped profile with a protruding portion 130 that complements a protruding portion of the connector 102. The second end 110B of the receiving ring 110 may include an opening 132. The receiving ring 110 may have an exterior surface 110C and an interior surface 110D. The receiving ring 110 may include one or more notches 144 formed along the exterior surface 110C.

As described above, the connector 102 may include the compressible member 116, also referred to as a silicone valve, disposed within the interior of the connector 102. The compressible member 116 may be disposed within the cavity 124 and extend through the receiving ring 110. The compressible member 116 have a first end 116A positioned proximate a surface of the wall 120 and a second end 116B positioned within the opening 132 of the receiving ring 110 and configured to engage a component of the connector 104. The second end 116B may include an engagement surface 134. The engagement surface 134 may provide a planar, or generally planar, surface that can be readily accessed for cleaning and disinfecting. The engagement surface 134 may further include an opening 136. In some embodiments, the opening 136 may be formed as one or more slits or apertures. The post 112 may extend through the compressible member 116. In other words, the compressible member 116 may extend around an exterior surface of the post 112. The second end 112B of the post 112 may be positioned within an area formed proximate the second end 112B of the compressible member 116.

In some embodiments, the compressible member 116 includes a bellow that can elastically compress. Accordingly, the compressible member 116 can compress by an external force and subsequently return to its original, uncompressed form when the external force is removed. The compressible member 116 may be designed to regulate fluid flow through the connector 102.

As illustrated in FIG. 2, the connector 104 may be a CSTD having a include a first end 104A configured to mate with the connector 102 when coupled and a second end 104B opposite the first end 104A. The connector 104 may include an exterior surface 104C and an interior surface 104D. The connector may include a channel 138 extending through the interior of the connector 104. The connector 104 may further include a spring member 166 disposed within the interior of the connector 104.

In some embodiments, the connector 104 may further include a connector interface 140 coupled to the second end 104B of the connector 104. The connector interface 140 may include a first end 140A configured to couple to the connector 104 and a second end 140B opposite the first end 140A. The connector interface 140 may be securing coupled to the second end 104B of the connector 104 by any suitable means to prevent preserve the closed state of the connector 104. For example, the connector interface 140 may be threadedly coupled to the second end 104B. The connector interface 140 may include a channel 142 extending through the connector interface 140 and providing a passageway for a needle 20. In some embodiments, the connector interface 140 may provide a channel 142 that allows for the needle 20 to be completely enclosed thereby preventing needle exposure and accidental needlesticks. The connector interface 140 may include an exterior surface 140C and an interior surface 140D. The exterior surface 140C may further include one or more projections 146 extending radially from the exterior surface 140C.

The connector 104 may further include a snap member 148, also referred to as a snap release. The snap member 148 may be disposed at the first end 104A of the connector 104. The snap member 148 may include a first end 148A, a second end 148B opposite the first end, an exterior surface 148C, and an interior surface 148D. The snap member 148 may include a plurality of snap arms 150 positioned on the exterior surface 148C of the snap member 148. In some embodiments, the snap arms 150 may be integrally formed with the snap member 148. In other embodiments, the snap arms 150 may be individually formed separate from a body of the snap member 148 and coupled via any suitable means. The snap arms 150 may include a first end 150A coupled to, or integrally formed with, the body of the snap member, and a second end 150B opposite the first end 150A. The second end 150B may be positioned proximate but spaced away from the exterior surface 104C of the connector 104 such that a gap 152 is formed between an interior surface of the snap arm 150 and the exterior surface 104C of the connector 104. The snap arms 150 may be flexible such the second end 150B may flex inwardly with respect to the exterior surface 104C, thereby decreasing the size of the gap 152, when decoupling the fluid connector assembly 100. The first end 148A of the snap arm may include an opening 156 for receiving a connecting mechanism 200, 300, 400, 500. The opening 156 may include sides 158 that form the perimeter of the opening 156. The sides 158 may be shaped and sized to correspond a portion of the connecting mechanism 200, 300, 400, 500. Furthermore, the sides 158 may be formed with a profile having one or more notches or depressions for receiving the connecting mechanism 200, 300, 400, 500, which will be described in greater detail below.

The connector 104 may include a valve assembly 160 coupled to the first end 104A of the connector 104. The valve assembly 160 may include a compartment 162 having one or more compressible members, also referred to as elastomeric plugs, 164 disposed within the compartment 162. The valve assembly 160 may be sized and shaped to completely fill the opening formed at the first end 104A of the connector 104. In some embodiments, the valve assembly 160 is integrally formed with the connector 104. In other embodiments, the valve assembly 160 is separately formed and coupled to the connector 104 via any suitable means to maintain a closed system. For example, the valve assembly 160 may be threadedly coupled. The valve assembly 160 may be coupled at an opposing end to the snap member 148. In some embodiments, the valve assembly 160 is substantially housed within the snap member 148.

As described above, the valve assembly 160 may include one or more compressible members 164. In some embodiments, the valve assembly 160 a first compressible member 164A and a second compressible member 164B housed within an interior cavity of the compartment 162. The first compressible member 164A may be positioned proximate the snap member 148 and the second compressible member 164B may be positioned opposite the first compressible member 164A. Fluid flowing through the fluid inlet 106 may pass through the compressible members 164A, 164B via the apertures formed within the compressible members 164A, 164B. Furthermore, as illustrated in FIG. 2, the compressible members 164A, 164B may be configured to allow the needle 20 to pass through the valve assembly 160 to provide drug delivery through the connector 104. In some embodiments, each of the compressible members 164A, 164B includes a bellow that can elastically compress. Accordingly, the compressible members 164A, 164B can compress by an external force and subsequently return to their original, uncompressed form when the external force is removed. The compressible members 164A. 164B are designed to regulate fluid flow through the connector 104.

Based on the position of the compressible members 164A, 164B shown in FIG. 2, the connector 104 may prevent fluid flow therethrough. In other words, fluid that enters via the fluid inlet 106, may be prevented from passing through the due to the positioning and expanded state of the compressible members 164A, 164B.

In order to promote fluid through the connectors 102, 104, when the fluid connector assembly 100 is assembled, the respective compressible members 116, 164A, 164B may be actuated, or displaced, to uncover respective openings in the connectors 102, 104.

In some embodiments, a fluid, including a medical fluid, can pass through the fluid connector assembly 100 downstream from the connector 104 to the connector 102 within a closed system. The fluid can enter the fluid inlet 106 and subsequently pass through the compressible members 164A, 164B. The fluid can then pass through the opening 128 of the post 112, and then through the channel 126 of the post 112. The fluid can exit the fluid connector assembly 100 through the fluid outlet 108.

As described above, the decoupling of the connectors 102 and 104 may result from an external force applied to one or more of the connectors 102 and 104, where the external force exceeds the threshold force required to maintain a connection between the connectors 102 and 104.

When the connectors 102, 104 are decoupled, the force applied to the compressible members 116, 164 of the connectors 102, 104, respectively, may be removed such that the compressible members 116, 164 may decompress and expand such that the compressible members 116, 164 return to their original shapes. For example, during expansion of the compressible member 116, the second end 116B of the compressible member 116 may move toward the second end 110B of the receiving ring 110 until the compressible member 116 has returned to its original shape such of the and the second end 116B is positioned within the opening 132 of the receiving ring 110. Similarly, during expansion of the compressible member 164, the first end 160A may move toward the first end 104A until the compressible member 164 has returned to its original shape such that the first end is positioned within the opening of the connector 104. As a result, fluid may be unable to flow upstream through the connector 102 and pass through the openings formed in the post 112.

Each of the compressible members 116 and 164 may be used as a valve to regulate fluid through the fluid connector assembly 100. Based on their elastic, springlike properties, each of the compressible members 116 and 164 may automatically return to their respective shapes when external forces are no longer acting on them. Beneficially, the compressible members 116 and 164 may spring back in response to a decoupling between the connectors 102 and 104 to provide a relatively quick sealing effect. Accordingly, blood losses, IV fluid losses, and medical delivery delays can be limited or prevented, based on one or more properties of the compressible members 116 and 164, when integrating the fluid connector assembly 100 into an IV set and catheter.

In some embodiments, the fluid connector assembly 100 may further include a connecting mechanism 200, 300, 400, 500 for coupling the connectors 102, 104.

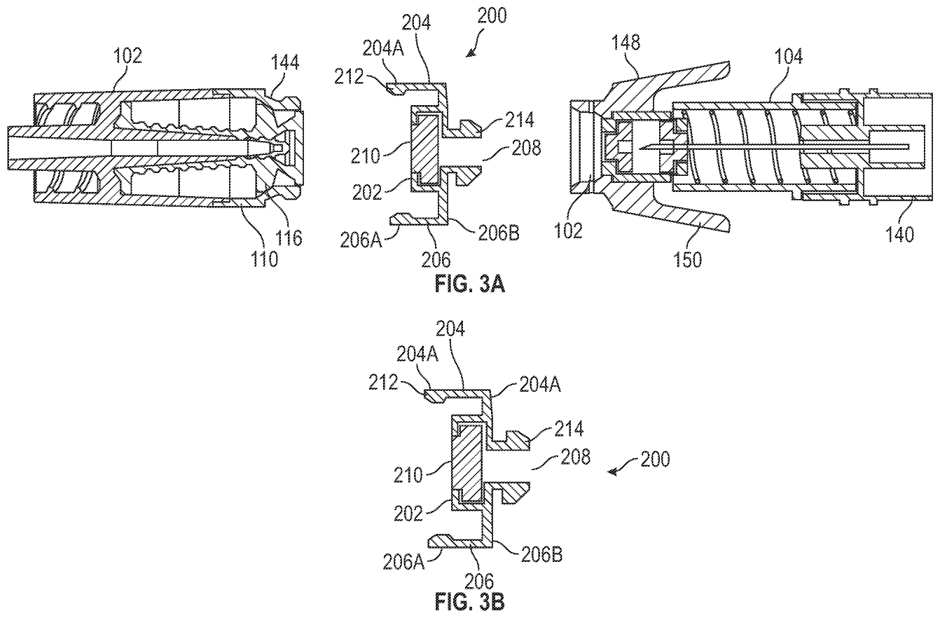

Referring to FIG. 3, the connecting mechanism 200 may include a body 202 comprised of two halves 204, 206, also referred to first and second sides, respectively. Each of the first side 204 and the second side 206 may include a first end 204A, 206B, respectively, and a second end 204B, 206B, respectively, opposite the first end 204A, 206B. The body 202 may further include a channel 208 extending therethrough for providing fluid communication between the connectors 102, 104. The connecting mechanism 200 may include a valve, also referred to as a plug member, 210 disposed within the channel 208. The valve 210 may regulate the flow of fluids through the channel 208. For example, when the connectors 102, 104 are coupled, the valve 210 may allow the flow of fluids through the channel 208. Conversely, when the connectors 102, 104 are not coupled, the valve 210 may prevent the flow of fluids through the channel 208. The connecting mechanism 200 may further comprise one or more projections 212, 214. In some embodiments, each of the first side 204 and the second side 206 includes a first projection 212 disposed on the first end 204A, 206A and a second projection 214 disposed on the second end 204B, 206B. The first projection 212 may be configured to couple to the connector 102. The first projection 212 may include an inwardly extending portion that projects toward the longitudinally extending central axis of the connecting mechanism 200. The inwardly extending portion of the first projection 212 may be sized and shaped to correspond to the notches 144 of the receiving ring to couple the connecting mechanism 200 to the connector 104. The second projection 214 may include an outwardly extending portion that projects away from the longitudinally extending central axis of the connecting mechanism 200. The outwardly extending portion may be sized and shaped to correspond to the sides 158 of the snap member 148.

To decouple the connecting mechanism 200 from the connector 104, a user may actuate the snap arms 150 by applying a pressing force to the snap arms 150 thereby providing clearance between the second projections 214 and the interior of the sides 158 of the snap member 148. When a sufficient clearance has been provided, a user may remove the connecting mechanism 200 from the connector 102.

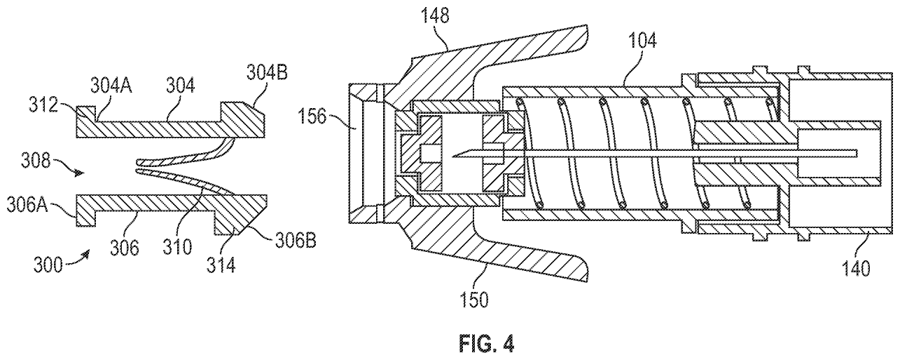

Referring to FIG. 4, the fluid connecting assembly may include a connecting mechanism 300. Similar to connecting mechanism 200, described above, connecting mechanism 300 may include a body 302 having two halves, also referred to as a first side 304 and a second side 306. Each of the first side 304 and the second side 306 may include a first end 304A, 306B, respectively, and a second end 304B, 306B, respectively, opposite the first end 304A, 306B. The body 302 may further include a channel 308 extending therethrough for providing fluid communication between the connectors 102, 104. The connecting mechanism 300 may include a valve, also referred to as a one-way valve, 310 disposed within the channel 308. The valve 310 may regulate the flow of fluids through the channel 308. For example, when the connectors 102, 104 are coupled, the valve 310 may allow the flow of fluids through the channel 308. Conversely, when the connectors 102, 104 are not coupled, the valve 310 may prevent the flow of fluids through the channel 308. The connecting mechanism 300 may further comprise one or more projections 312, 314. In some embodiments, each of the first side 304 and the second side 306 includes a first projection 312 disposed on the first end 304A, 306A and a second projection 314 disposed on the second end 304B, 306B. The first projection 312 may be configured to couple to the connector 102. The first projection 312 may include an outwardly extending portion that projects toward the longitudinally extending central axis of the connecting mechanism 300. The first projection 312 may be sized and shaped to correspond to the opening 132 of the receiving ring 110. When the connecting mechanism 300 is coupled to the connector 102, an end of the first projection 312 may contact the compressible member 116. The first projection may aid in compressing the compressible member 116 to allow for the fluid flow. The second projection 314 may include an outwardly extending portion that projects away from the longitudinally extending central axis of the connecting mechanism 300. The outwardly extending portion may be sized and shaped to correspond to the sides 158 of the snap member 148.

As described above with respect to connecting mechanism 200, to decouple the connecting mechanism 300 from the connector 104, a user may actuate the snap arms 150 by applying a pressing force to the snap arms 150 thereby providing sufficient clearance between the second projections 314 and the interior of the sides 158 of the snap member 148.

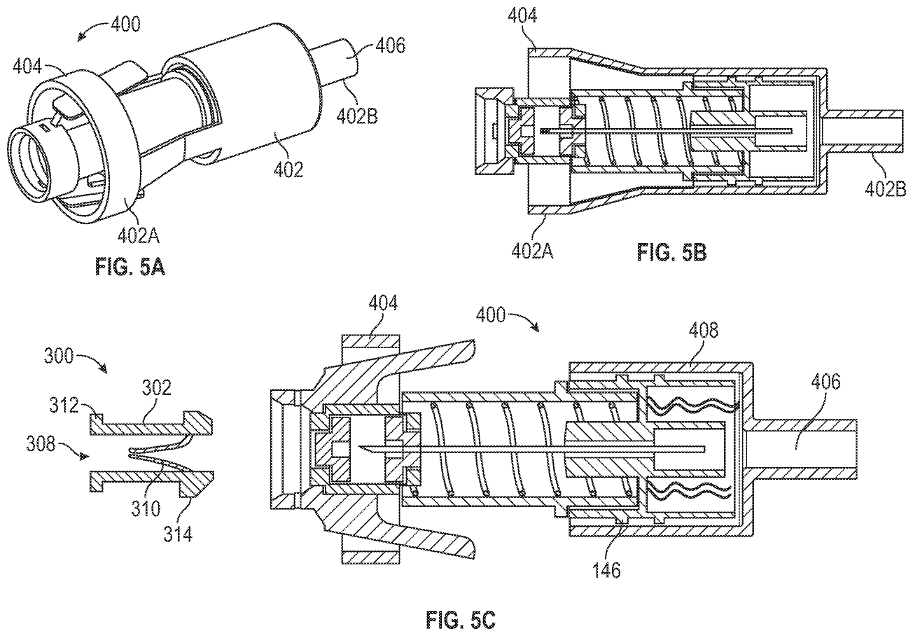

Referring to FIGS. 5A-5C, the connecting mechanism 400 may include a body 402 disposed on the exterior surface 104C of the connector 104. The connecting mechanism 400 may include a first end 402A disposed proximate the snap member 148 and a second end 402B opposite the first end 402A and disposed proximate the connector interface 140. The first end 402A may include a ring 404 that encompasses at least a portion of the snap arms 150. The ring 404 may be sized such that only a slight gap exists between an interior surface of the ring 404 and an exterior surface of the snap arm 150. The second end 402B may include a bond pocket 406 and may encompass the connector interface 140. The bond pocket 406 may be coupled to tubing 22 of the IV set 1. The bond pocket 406 may be coupled via any suitable means, including, but not limited to gluing. The body 402 may further include one or more notches or detents 408 to mate with projections 146 of the connector interface 140 thereby further securing the connecting mechanism 400 to the connector 104. In some embodiments, a substantial portion of the connector 104 is not encompassed by the connecting mechanism 400. The connecting mechanism 400 may be configured to slide a certain distance along the connector 104.

The connecting mechanism 400 may work in conjunction with connecting mechanism 300. When assembled, the connecting mechanism 400 is secured to the connector 104 with the ring 404 disposed proximate the first end 148A of the snap member 148. Meanwhile, the connecting mechanism 300 may be coupled to the connectors 102, 104 as described above, thereby providing fluid communication between the connectors 102, 104. In the event that the tubing 22 is pulled with sufficient force in a direction away from the second end 104B of the connector 104, the connecting mechanism 400 may slide relative to the connector 104. In particular, the ring 404 may slide in a direction along the snap arm 150 from the first end 150A to the second end 150B. As the snap arms 150 are angled in a direction away from the central longitudinal axis of the connector 104, the ring 404 may slide until the interior surface of the ring 404 abuts the exterior surface of the snap arm 150 and is prevented from moving further due to friction. When the ring 404 reaches this stopping point, which may be proximate the second end 150B, pressure exerted from the ring 404 may compress the snap arm 150 thereby decoupling the connecting mechanism 300 from the opening 156 of the snap member 148.

Should a user desire to purposefully decouple the connecting mechanism 300 from the connector 104, a user may perform the steps described above with respect to the connecting mechanism 200, 300. In other words, a user may actuate the snap arms 150 by applying a pressing force to the snap arms 150 thereby providing sufficient clearance between the second projections 314 and the interior of the sides 158 of the snap member 148. Alternatively, a user may pull the connecting mechanism 400 from a position proximate the second end 402B until the ring 404 provides sufficient force to the snap arm 150 to decouple the connecting mechanism 300.

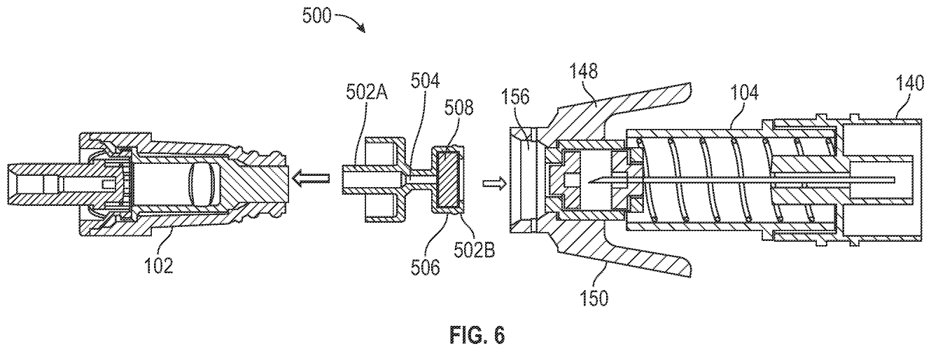

Referring to FIG. 6, the connecting mechanism 500 may include a body 502 having a first end 502A configured to couple to the connector 102 and a second end 502B, opposite the first end 502A and configured to couple to the connector 104. The body 502 may further include a channel 504 extending therethrough for providing fluid communication between the connectors 102, 104. The channel 504 may include a forward portion 506 for housing a valve, also referred to as a plug member, 508 disposed within the forward portion 506. As described above, with respect to the connecting mechanism 200, the valve 508 may regulate the flow of fluids through the channel 504. The connecting mechanism 500 may further include a projection 510 disposed at the second end 502B. The projection 510 may be formed as a male luer and configured to contact and couple to the connector 102. When the connecting mechanism 500 is coupled to the connector 102, an end of the projection 510 may contact the compressible member 116. The first projection may aid in compressing the compressible member 116 to allow for the fluid flow. The forward portion 506 may be sized and shaped to be received within the opening 156 of the snap member 148.

The connecting mechanism 500 may be decoupled in the same manner described above with respect to connecting mechanism 200, 300, 400. In other words, a user may apply a pressing force to the snap arms 150 to provide sufficient clearance to remove the connecting mechanism 500 from within the snap member.

In use, the connecting mechanism 200, 300, 400, 500 and connector 104 may remain coupled until acted upon by a decoupling force. The decoupling force may pull connector 102 away from connector 104 and the connecting mechanism 200, 300, 400, 500 or pull connector 104 and the connecting mechanism 200, 300, 400, 500 away from connector 102. The decoupling force may act along an axis that is central to connectors 102, 104, and the connecting mechanism 200, 300, 400, 500 while they are coupled. The decoupling force may be a resultant force acting along an axis central to the connectors 102, 104, and connecting mechanism 200, 300, 400, 500 while they are coupled, or of a force that acts upon connectors 102, 104 or upon the connecting mechanism 200, 300, 400, 500, or upon the first portion or tubing or upon the second portion of tubing.

The decoupling force may decouple the connecting mechanism 200, 300, 400, 500 from connector 104 when decoupling force exceeds a predetermined threshold force. For example, if the decoupling force is less than the predetermined threshold force, the connecting mechanism 200, 300, 400, 500 may not decouple from connector 104. The predetermined threshold force prevents inadvertent or accidental decoupling based on minor forces or movements. When the connectors 102, 104 are inadvertently decoupled, the connecting mechanism 200, 300, 500 may remain coupled to the connector 102.

In some embodiments, the predetermined threshold force is approximately 5 pounds (lbs). The predetermined threshold force may be from approximately 1 lb to approximately 8 lbs, approximately 3 lbs to approximately 7 lbs, approximately 4 lbs to approximately 6 lbs, or greater than 8 lbs.

The features of the present disclosure provide first and second compressible members that can be used as valves to regulate a fluid pathway therebetween. The first and second compressible members are located in a first and a second connector, respectively. If the first and second connectors are separated, whether unintentionally or intentionally, the fluid pathway for each of the first and second compressible members become closed or obstructed to prevent fluid loss therefrom. The features of the present disclosure also provide that upon separation of the first and second compressible members, any of the first and second compressible members can be cleaned and disinfected, and the first and second compressible members can be once again coupled together to form a fluid pathway therebetween.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 11, clause 17, clause, clause 23, clause 26, or clause 29. The other clauses can be presented in a similar manner.

Clause 1. A fluid connector assembly, comprising: a first connector; a second connector configured to couple with the first connector, the second connector having a snap member disposed on an end of the second connector; and a connecting mechanism coupled to the second connector for detachably coupling the first connector and the second connector, the connecting mechanism comprising: a body having a channel extending therethrough for providing fluid communication between the first connector and the second connector; and at least one projection extending from an end of the body, wherein, when the connecting mechanism is coupled to the second connector, at least a portion of the connecting mechanism is positioned within an opening of the snap member and secured by the at least one projection, and wherein actuation of the snap member allows the connecting mechanism to be disconnected from the second connector.

Clause 2. The fluid connector assembly described above, wherein the connecting mechanism remains coupled to the second connector by a threshold force, and wherein, when an external force greater than the threshold force is applied to at least one of the first connector and the second connector, the connecting mechanism is decoupled from the second connector.

Clause 3. The fluid connector assembly described above, wherein the external force is a pulling force of at least five pounds.

Clause 4. The fluid connector assembly described above, wherein the snap member includes at least one snap arm extending from an end of the snap member, and wherein actuation of the at least one snap arm allows for the connecting mechanism to be decoupled from the second connector.

Clause 5. The fluid connector assembly described above, wherein the connecting mechanism further comprises a valve member disposed within the channel, and wherein, when the first connector and the second connector are coupled, the valve member is configured to allow fluid communication between the first connector and the second connector, and wherein, when the first connector and the second connector are decoupled, the valve member is configured to prevent a flow of a fluid through the channel.

Clause 6. The fluid connector assembly described above 1, wherein the at least one projection extends outwardly with respect to a longitudinally extending central axis, and wherein an exterior surface profile of the at least one projection corresponds to an interior profile of the opening of the snap member.

Clause 7. The fluid connector assembly described above, wherein at least a portion of the connecting mechanism couples to an exterior surface of the second connector, wherein when a pulling force greater than a threshold force is applied to the second connector in a direction along a central longitudinal axis, the connecting mechanism actuates the snap member thereby resulting, and wherein at least a portion of the connecting mechanism remains coupled to the second connector when the first connector and the second connector are decoupled.

Clause 8. The fluid connector assembly described above, wherein the fluid connector assembly is a closed-system drug transfer device the at least one projection comprises: a first projection extending from a first end of the body of the connecting mechanism for coupling to the first connector; and a second projection extending from a second end of the body of the connecting mechanism for coupling to the second connector. Clause 9. A method of coupling a fluid connector assembly, the method comprising:

providing a first connector; providing a second connector configured to couple to the first connector, the second connector having a snap member disposed on an end of the second connector; providing a connecting mechanism configured to couple the first connector and the second connector, the connecting mechanism comprising a body and at least one projection extending from an end of the body; coupling an end of the connecting mechanism to the second connector such that at least a portion of connecting mechanism is disposed within an opening of the snap member; and coupling the first connector to an opposite end of the connecting mechanism such that the first connector, the second connector, and coupling mechanism are in fluid communication.

Clause 10. The method described above, wherein coupling the end of the connecting mechanism to the second connector includes positioning the at least one projection within the opening of the snap member such that an exterior surface of the at least one projection abuts an interior surface of the snap member in a snap fit configuration.

Clause 11. The method described above, wherein decoupling the connecting mechanism from the second connector includes actuating the snap member to provide clearance between the at least one projection and the interior surface thereby allowing the connecting mechanism to be removed from the opening.

Clause 12. The method described above, wherein the connecting mechanism remains coupled to the second connector by a threshold force, and wherein, when an external force greater than the threshold force is applied to at least one of the first connector and the second connector, the connecting mechanism is decoupled from the second connector.

Clause 13. The method described above, wherein the connecting mechanism further comprises a valve member disposed within a channel formed within the body of the connecting mechanism, wherein, when the first connector and the second connector are coupled, the valve member is configured to allow fluid communication between the first connector and the second connector, and wherein, when the first connector and the second connector are decoupled, the valve member is configured to prevent a flow of a fluid through the channel.

Clause 14. A fluid connector assembly comprising: a first connector; a second connector configured to couple to the first connector, the second connector is a closed system comprising a snap member disposed having at least one arm projecting from an end of the snap member; a connecting mechanism for detachably coupling the first connector and the second connector, the connecting mechanism comprising: a body having a channel extending therethrough for providing fluid communication between the first connector and the second connector; a valve member disposed within the channel for controlling a flow of fluids through the channel; a first projection extending from a first end of the body for coupling to the first connector; and a second projection extending from a second end of the body for coupling to the second connector, wherein, when the connecting mechanism is coupled to the second connector, at least a portion of the connecting mechanism is positioned within an opening of the snap member and secured by the second projection, and wherein, when a separating force exceeds a predetermined threshold, the connecting mechanism is decoupled from the second connector.

Clause 15. The fluid connector assembly described above, wherein the predetermined threshold is a pulling force of at least five pounds.

Clause 16. The fluid connector assembly described above, wherein the at least one arm includes a first snap arm projecting from a first side of the second connector and a second snap arm projecting from a second side of the second connector opposite the first side, and wherein actuation of the first snap arm and the second snap arm allows for the connecting mechanism to be decoupled from the second connector.

Clause 17. The fluid connector assembly described above, wherein, when the first connector and the second connector are coupled, the valve member is configured to allow fluid communication between the first connector and the second connector, and wherein, when the first connector and the second connector are decoupled, the valve member is configured to prevent a flow of a fluid through the channel.

Clause 18. The fluid connector assembly described above, wherein the each of the first projection and the second projection extends outwardly with respect to a longitudinally extending central axis of the connecting mechanism, and wherein an exterior surface profile of the second projection corresponds to an interior profile of the opening of the snap member.

Clause 19. The fluid connector assembly described above, wherein the connecting mechanism includes a ring member slidably disposed on an exterior surface of the second connector and configured to actuate the snap member.

Clause 20. The fluid connector assembly described above, wherein, when a separating force exceeds a predetermined threshold, the ring member slides along the second connector until the ring member is disposed on the at least one arm thereby causes actuation of the snap member.

### FURTHER CONSIDERATIONS

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents.

Notwithstanding, none of the claims are intended to embrace subject matter that fails to satisfy the requirement of 35 U.S.C. § 101, 102, or 103, nor should they be interpreted in such a way.

What is claimed is:

1. A fluid connector assembly, comprising:
a first connector;
a second connector configured to couple with the first connector, the second connector having a snap member disposed on an end of the second connector; and
a connecting mechanism coupled to the second connector for detachably coupling the first connector and the second connector, the connecting mechanism comprising:
a body having a channel extending therethrough for providing fluid communication between the first connector and the second connector; and
at least one projection extending from an end of the body,
wherein, when the connecting mechanism is coupled to the second connector, at least a portion of the connecting mechanism is positioned within an opening of the snap member and secured by the at least one projection, and
wherein actuation of the snap member allows the connecting mechanism to be disconnected from the second connector.

2. The fluid connector assembly of claim 1, wherein the connecting mechanism remains coupled to the second connector by a threshold force, and
wherein, when an external force greater than the threshold force is applied to at least one of the first connector and the second connector, the connecting mechanism is decoupled from the second connector.

3. The fluid connector assembly of claim 2, wherein the external force is a pulling force of at least five pounds.

4. The fluid connector assembly of claim 1, wherein the snap member includes at least one snap arm extending from an end of the snap member, and
wherein actuation of the at least one snap arm allows for the connecting mechanism to be decoupled from the second connector.

5. The fluid connector assembly of claim 1, wherein the connecting mechanism further comprises a valve member disposed within the channel, and
wherein, when the first connector and the second connector are coupled, the valve member is configured to allow fluid communication between the first connector and the second connector, and
wherein, when the first connector and the second connector are decoupled, the valve member is configured to prevent a flow of a fluid through the channel.

6. The fluid connector assembly of claim 1, wherein the at least one projection extends outwardly with respect to a longitudinally extending central axis, and
wherein an exterior surface profile of the at least one projection corresponds to an interior profile of the opening of the snap member.

7. The fluid connector assembly of claim 1, wherein at least a portion of the connecting mechanism couples to an exterior surface of the second connector,
wherein when a pulling force greater than a threshold force is applied to the second connector in a direction along a central longitudinal axis, the connecting mechanism actuates the snap member thereby resulting in the first connector and second connector being decoupled, and
wherein at least a portion of the connecting mechanism remains coupled to the second connector when the first connector and the second connector are decoupled.

8. The fluid connector assembly of claim 1, wherein the fluid connector assembly is a closed-system drug transfer device.

9. A method of coupling a fluid connector assembly, the method comprising:
providing a first connector;
providing a second connector configured to couple to the first connector, the second connector having a snap member disposed on an end of the second connector;
providing a connecting mechanism configured to couple the first connector and the second connector, the connecting mechanism comprising a body and at least one projection extending from an end of the body;
coupling an end of the connecting mechanism to the second connector such that at least a portion of connecting mechanism is disposed within an opening of the snap member; and
coupling the first connector to an opposite end of the connecting mechanism such that the first connector, the second connector, and coupling mechanism are in fluid communication.

10. The method of claim 9, wherein coupling the end of the connecting mechanism to the second connector includes positioning the at least one projection within the opening of the snap member such that an exterior surface of the at least one projection abuts an interior surface of the snap member in a snap fit configuration.

11. The method of claim 10, wherein decoupling the connecting mechanism from the second connector includes actuating the snap member to provide clearance between the at least one projection and the interior surface thereby allowing the connecting mechanism to be removed from the opening.

12. The method of claim 9, wherein the connecting mechanism remains coupled to the second connector by a threshold force, and
wherein, when an external force greater than the threshold force is applied to at least one of the first connector and the second connector, the connecting mechanism is decoupled from the second connector.

13. The method of claim 9, wherein the connecting mechanism further comprises a valve member disposed within a channel formed within the body of the connecting mechanism,
wherein, when the first connector and the second connector are coupled, the valve member is configured to allow fluid communication between the first connector and the second connector, and
wherein, when the first connector and the second connector are decoupled, the valve member is configured to prevent a flow of a fluid through the channel.

14. A fluid connector assembly comprising:
a first connector;
a second connector configured to couple to the first connector, the second connector is a closed system comprising a snap member disposed having at least one arm projecting from an end of the snap member;
a connecting mechanism for detachably coupling the first connector and the second connector, the connecting mechanism comprising:
a body having a channel extending therethrough for providing fluid communication between the first connector and the second connector;
a valve member disposed within the channel for controlling a flow of fluids through the channel;
a first projection extending from a first end of the body for coupling to the first connector; and
a second projection extending from a second end of the body for coupling to the second connector,
wherein, when the connecting mechanism is coupled to the second connector, at least a portion of the connecting mechanism is positioned within an opening of the snap member and secured by the second projection, and
wherein, when a separating force exceeds a predetermined threshold, the connecting mechanism is decoupled from the second connector.

15. The fluid connector assembly of claim 14, wherein the predetermined threshold is a pulling force of at least five pounds.

16. The fluid connector assembly of claim 14, wherein the at least one arm includes a first snap arm projecting from a first side of the second connector and a second snap arm projecting from a second side of the second connector opposite the first side, and
wherein actuation of the first snap arm and the second snap arm allows for the connecting mechanism to be decoupled from the second connector.

17. The fluid connector assembly of claim 14, wherein, when the first connector and the second connector are coupled, the valve member is configured to allow fluid communication between the first connector and the second connector, and
wherein, when the first connector and the second connector are decoupled, the valve member is configured to prevent a flow of a fluid through the channel.

18. The fluid connector assembly of claim 14, wherein the each of the first projection and the second projection extends outwardly with respect to a longitudinally extending central axis of the connecting mechanism, and
wherein an exterior surface profile of the second projection corresponds to an interior profile of the opening of the snap member.

19. The fluid connector assembly of claim 14, wherein the connecting mechanism includes a ring member slidably disposed on an exterior surface of the second connector and configured to actuate the snap member.

20. The fluid connector assembly of claim 19, wherein, when a separating force exceeds a predetermined threshold, the ring member slides along the second connector until the ring member is disposed on the at least one arm thereby causes actuation of the snap member.
